(19) United States
(12) Patent Application Publication
(10) Pub. No.: US 2022/0306609 A1
(43) Pub. Date: Sep. 29, 2022

(54) BICYCLIC COMPOUNDS AS ANDROGEN RECEPTOR MODULATORS

(71) Applicant: NIDO BIOSCIENCES, INC., Cambridge, MA (US)

(72) Inventors: Bakary-Barry TOURE, WESTON, MA (US); Mark Andrew GALLOP, SAN FRANCISCO, CA (US); Hanmo ZHANG, Watertown, MA (US)

(21) Appl. No.: 17/701,888

(22) Filed: Mar. 23, 2022

Related U.S. Application Data
(60) Provisional application No. 63/164,872, filed on Mar. 23, 2021.

(51) Int. Cl. C07D 403/04; C07D 471/04

(52) U.S. Cl. CPC C07D 403/04; C07D 471/04

(57) ABSTRACT

Provided herein are indole compounds that bind to BF3 of an androgen receptor (AR), which can modulate the AR for the treatment of Kennedy's disease

### RELATED APPLICATIONS

This application claims priority to U.S. Provisional Application No. 63/164,872 filed Mar. 23, 2021, the entire content of which is hereby incorporated by reference in its entirety.

### BACKGROUND

Prostate cancer is the second leading cause of male cancer-related death in Western countries (Damber, J. E. and Aus, G. Lancet (2008) 371:1710-1721). Numerous studies have shown that the androgen receptor (AR) is central not only to the development of prostate cancer, but also the progression of the disease to the castration resistance state (Taplin, M. E. et al. J. Clin. Oncol. (2003) 21:2673-8; and Tilley, W. D. et al. Cancer Res. (1994) 54:4096-4102). Thus, effective inhibition of human AR remains one of the most effective therapeutic approaches to the treatment of advanced, metastatic prostate cancer.

Androgen receptor activity is also liked to Kennedy's disease, also referred to as Spinal Bulbar Muscular Atrophy (SBMA). Kennedy's disease is an x-linked recessive motor neuron disease resulting from disruptions in the transmission of nerve cell signals in the brain stem and spinal cord. The motor neuron disruptions are more noticeable relative to other cells because of the higher number of the androgen receptors residing in nerve cells. The nerve cells in a Kennedy's patient gradually become increasingly dysfunctional and eventually die, leaving the muscles unable to contract, resulting in atrophy of the muscles throughout the body, but most noticeably in the extremities, face and throat. The binding of testosterone to the AR is thought to cause the disease.

There remains a need for effective treatments for both prostate cancer and Kennedy's disease.

### SUMMARY

Provided herein are compounds that modulate androgen receptor (AR) activity. In particular, the compounds disclosed herein show inhibition of Androgen Receptor Binding Function-3 (BF3).

In an aspect, provided herein is a compound of Formula I:

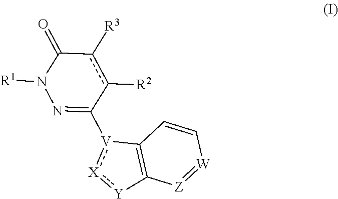

or a pharmaceutically acceptable salt thereof; wherein the variables are defined herein.

In another aspect, provided herein is a compound of Formula II

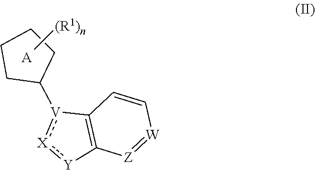

or a pharmaceutically acceptable salt thereof; wherein the variables are defined herein.

In another aspect, provided herein is a pharmaceutical composition comprising a compound of Formula I and a pharmaceutically acceptable carrier.

In yet another aspect, provided herein is a method of treating a neurodegenerative disorder in a subject in need thereof comprising administering to the subject a therapeutically effective amount of a compound of Formula I. In an embodiment of the methods, the neurodegenerative disorder is spinal bulbar muscular atrophy (SBMA).

In still another aspect, provided herein is a method of modulating androgen receptor (AR) activity in a subject in need thereof comprising administering to the subject a therapeutically effective amount of a compound of Formula I.

In another aspect, provided herein is a method of treating cancer in a subject in need thereof comprising administering to the subject a therapeutically effective amount of a compound of Formula I. In an embodiment, the cancer is prostate cancer.

### DETAILED DESCRIPTION

Androgens play a role in a wide range of developmental and physiological responses, for example, male sexual differentiation, maintenance of spermatogenesis, and male gonadotropin regulation (Ross, R. K., et al., Eur. Urol. 35, 355-361 (1999); Thomson, A. A., Reproduction 121, 187-195 (2001); Tanji, N., et al., Arch. Androl. 47, 1-7 (2001)). Androgens are also associated with the development of prostate carcinogenesis. Induction of prostatic carcinogenesis in rodent models has been associated with androgens (R. L. Noble, Cancer Res. 37, 1929-1933 (1977); R. L. Noble, Oncology 34, 138-141 (1977)), and men receiving androgens in the form of anabolic steroids are reported to have a higher incidence of prostate cancer (Roberts, J. T., and Essenhigh, D. M., Lancet 2, 742 (1986); Jackson, J. A., et al., Arch. Intern. Med. 149, 2365-2366 (1989); Guinan, P. D., et al., Am. J. Surg. 131, 599-600 (1976)). Furthermore, prostate cancer does not develop if humans or dogs are castrated before puberty (Wilson, J. D., and Roehrborn, C., J. Clin. Endocrinol. Metab. 84, 4324-4331 (1999); G. Wilding, Cancer Surv. 14, 113-130 (1992)). Castration of adult males causes involution of the prostate and apoptosis of prostatic epithelium (Bruckheimer, E. M., and Kyprianou, N., Cell Tissue Res. 301, 153-162 (2000); J. T. Isaacs, Prostate 5, 545-557 (1984)). This dependency on androgens provides the underlying rationale for treating prostate cancer with chemical or surgical castration (i.e., androgen ablation).

The AR possesses a modular organization characteristic of all nuclear receptors. It is comprised of an N-terminal domain, a central DNA binding domain, a short hinge region, and C-terminal domain that contains a hormone ligand binding pocket and the Activation Function-2 (AF2) site (Gao, W. Q. et al. Chem. Rev. (2005) 105:3352-3370). The latter represents a hydrophobic groove on the AR surface which is flanked with regions of positive and negative charges, “charge clamps,” that are significant for binding AR activation factors (Zhou, X. E. et al. J. Biol. Chem. (2010) 285:9161-9171). Recent studies have identified a novel site on the AR called Binding Function 3 (BF3) that is involved into AR transcriptional activity.

It has been proposed that a small molecule bound to the BF3 site could cause the AR protein to undergo an allosteric modification that prevents AR interactions with co-activators. Importantly, the BF3 site is located near, but distinct from, the ligand-binding site that is normally targeted by conventional anti-androgen drugs. Compounds such as flufenamic acid (FLUF), thriiodothyronine (T3) and 3,3′,5-triiodo thyroacetic acid (TRIAC) can bind to the BF3 cleft, inhibit AF2 interactions, and interfere with AR activity (Estebanez-Perpina, E. et al. Proc. Natl. Acad. Sci. USA (2007) 104:16074-16079). While these compounds revealed the importance of the BF3 site, they have shown a low potency (IC₅₀>50 μM) and were found to bind non-specifically to the AR.

The activation of AR follows a well-characterized pathway: in the cytoplasm, the receptor is associated with chaperone proteins that maintain agonist binding conformation of the AR (Georget, V. et al. Biochemistry (2002) 41:11824-11831). Upon binding of an androgen, the AR undergoes a series of conformational changes, disassociation from chaperones, dimerization and translocation into the nucleus (Fang, Y. F. et al. J. Biol. Chem. (1996) 271:28697-28702; and Wong, C. I. et al. J. Biol. Chem. (1993) 268:19004-19012) where it further interacts with co-activator proteins at the AF2 site (Zhou, X. E. et al. J. Biol. Chem. (2010) 285:9161-9171). This event triggers the recruitment of RNA polymerase II and other factors to form a functional transcriptional complex with the AR.

Notably, the current anti-androgens such as bicalutamide, flutamide, nilutamide and MDV3100, all target this particular process. However, instead of affecting the AR-cofactor interaction directly, these anti-androgens act indirectly, by binding to the AR ligand binding site. Thus, by preventing androgens from binding they also prevent conformational changes of the receptor that are necessary for co-activator interactions. While treatment with these AR inhibitors can initially suppress prostate cancer growth, long term hormone therapy becomes progressively less effective (Taplin, M. E. et al. J. Clin. Oncol. (2003) 21:2673-8; and Tilley, W. D. et al. Cancer Res. (1994) 54:4096-4102). Factors that make the AR less sensitive to conventional anti-androgens include resistance mutations at the ligand binding site that can even lead AR antagonists to act as agonists further contributing to cancer progression (Chen, Y. et al. Lancet Oncol. (2009) 10:981-991).

Androgens also play a role in female cancers. One example is ovarian cancer where elevated levels of androgens are associated with an increased risk of developing ovarian cancer (K. J. Helzlsouer, et al., JAMA 274, 1926-1930 (1995); R. J. Edmondson, et al, Br J Cancer 86, 879-885 (2002)). The AR has been detected in a majority of ovarian cancers (H. A. Risch, J. Natl. Cancer Inst. 90, 1774-1786 (1998); B. R. Rao & B. J. Slotman, Endocr. Rev. 12, 14-26 (1991); G. M. Clinton & W. Hua, Crit. Rev. Oncol. Hematol. 25, 1-9 (1997)), whereas estrogen receptor-alpha (ERα) and the progesterone receptor are detected in less than 50% of ovarian tumors.

Spinal and bulbar muscular atrophy (SBMA), popularly known as Kennedy's disease, is a progressive debilitating neurodegenerative disorder resulting in muscle cramps and progressive weakness due to degeneration of motor neurons in the brainstem and spinal cord. The condition is associated with mutation of the androgen receptor (AR) gene and is inherited in an X-linked recessive manner. As with many genetic disorders, no cure is known, although research continues. Because of its endocrine manifestations related to the impairment of the AR gene, SBMA can be viewed as a variation of the disorders of the androgen insensitivity syndrome (AIS). It is also related to other neurodegenerative diseases caused by similar mutations, such as Huntington's disease.

The BF3 site is an attractive target for direct inhibition of the AR co-activation.

### Definitions

It is appreciated that certain features of the present disclosure, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula I can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group. The term “substituted” means that an atom or group of atoms formally replaces hydrogen as a “substituent” attached to another group. The term “substituted,” unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like. The term “alkyl” employed alone or in combination with other terms, refers to a saturated hydrocarbon group that may be straight-chained or branched. The term “Cₙ₋ₘ alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkoxy,” employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms. The term “Cₙ₋ₘ dialkoxy” refers to a linking group of formula —O—(Cₙ₋ₘ alkyl)-O—, the alkyl group of which has n to m carbons. Example dialkyoxy groups include —OCH₂CH₂O— and OCH₂CH₂CH₂O—. In some embodiments, the two O atoms of a Cₙ₋ₘ dialkoxy group may be attached to the same B atom to form a 5- or 6-membered heterocycloalkyl group. The terms “halo” or “halogen,” used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “aromatic” refers to a carbocycle or heterocycle having one or more polyunsaturated rings having aromatic character (i.e., having (4n+2) delocalized 0 (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl. In some embodiments, the aryl group is naphthyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, isoxazolyl, thiazolyl, imidazolyl, furanyl, thio-phenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, isoindolyl, benzothiophenyl, benzofuranyl, benzisoxazolyl, imidazo[1,2-b]thiazolyl, purinyl, and the like. In some embodiments, the heteroaryl group is pyridone (e.g., 2-pyridone).

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 2,5-diazabicyclo[2.2.1]-heptanyl; pyrrolidinyl; hexahydropyrrolo[3,4-b]pyrrol-1(2H)-yl; 1,6-dihydropyridinyl; morpholinyl; azetidinyl; piperazinyl; and 4,7-diazaspiro[2.5]octan-7-yl.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral center, each of the chiral centers in the compound may be independently (R) or (S), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et.al. J. Med. Chem. 2011, 54, 201-210; R. Xu et.al. J. Label Compd. Radiopharm. 2015, 58, 308-312).

The term “compound,” as used herein, is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds provided herein, or salts thereof, are substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selecton, and Use, (Wiley, 2002). In some embodiments, the compounds described herein include the N-oxide forms.

In some embodiments, pharmaceutical compositions as described herein may comprise a salt of such a compound, preferably a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents (used interchangeably herein) are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

An “effective amount” of a pharmaceutical composition as described herein includes a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to an androgen-independent form. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

Compounds as described herein may be administered to a subject. As used herein, a “subject” may be a human, non-human primate, rat, mouse, cow, horse, pig, sheep, goat, dog, cat, etc. In an embodiment, the subject is human.

Definitions used include ligand-dependent activation of the androgen receptor (AR) by androgens such as dihydrotestosterone (DHT) or the synthetic androgen (R1881) used for research purposes. Ligand-independent activation of the AR refers to transactivation of the AR in the absence of androgen (ligand) by, for example, stimulation of the cAMP-dependent protein kinase (PKA) pathway with forskolin (FSK).

Some compounds and compositions as described herein may interfere with a mechanism specific to ligand-dependent activation (e.g., accessibility of the ligand binding domain (LBD) to androgen) or to ligand-independent activation of the AR.

Various alternative embodiments and examples of the invention are described herein. These embodiments and examples are illustrative and should not be construed as limiting the scope of the invention.

### Compounds

In an aspect, provided herein is a compound of Formula I:

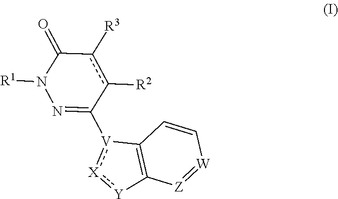

or a pharmaceutically acceptable salt thereof;

wherein

[symbol] is an optional double bond;

V is C, CH, or N;

X is CH or N;

Y is CH, N, NH, O, or S;

Z and W are each independently CR⁴ or N;

R¹ is selected from the group consisting of H, C₁-C₆ alkyl, C₁-C₆ haloalkyl, C₃-C₁₀ cycloalkyl, 3-10 membered heterocycloalkyl, C₆-C₁₀ aryl, and 5-10 membered heteroaryl;

R² is selected from the group consisting of H, C₁-C₆ alkyl, C₁-C₆ haloalkyl, C₁-C₆ alkoxy, C₃-C₁₀ cycloalkyl, 3-10 membered heterocycloalkyl, C₆-C₁₀ aryl, and 5-10 membered heteroaryl;

R³ is selected from the group consisting of H, C₁-C₆ alkyl, C₁-C₆ haloalkyl, C₁-C₆ alkoxy, C₃-C₁₀ cycloalkyl, 3-10 membered heterocycloalkyl, C₆-C₁₀ aryl, and 5-10 membered heteroaryl; and

R⁴ is independently, at each occurrence, selected from the group consisting of H, halo, CN, OH, NH₂, NH(C₁-C₆ alkyl), N(C₁-C₆ alkyl)₂, C₁-C₆ alkyl, C₁-C₆ haloalkyl, C₁-C₆ alkyl-OH, and C₁-C₆ alkoxy.

In an embodiment, the compound of Formula I is a compound of Formula Ia:

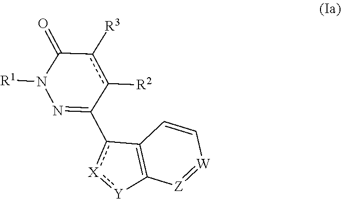

or a pharmaceutically acceptable salt thereof.

In an embodiment

[symbol] is an optional double bond;

V is CH or C;

X is CH or N;

Y is CH, N, NH, O, or S;

Z and W are each independently CR⁴ or N;

R¹ is selected from the group consisting of H, C₁-C₆ alkyl, C₁-C₆ haloalkyl, C₃-C₁₀ cycloalkyl, 3-10 membered heterocycloalkyl, C₆-C₁₀ aryl, and 5-10 membered heteroaryl;

R² is selected from the group consisting of H, C₁-C₆ alkyl, C₁-C₆ haloalkyl, and C₁-C₆ alkoxy;

R³ is selected from the group consisting of H, C₁-C₆ alkyl, C₁-C₆ haloalkyl, and C₁-C₆ alkoxy; and

R⁴ is independently, at each occurrence, selected from the group consisting of H, halo, CN, C₁-C₆ alkyl, C₁-C₆ haloalkyl, and C₁-C₆ alkoxy.

In an embodiment,

[symbol] is an optional double bond;

V is C or CH;

X is CH;

Y is NH;

Z and W are each independently CH, CR⁴, or N;

R¹ is C₁-C₆ alkyl or C₁-C₆ haloalkyl;

R² is H or C₁-C₆ alkyl;

R³ is selected from the group consisting of H, C₁-C₆ alkyl, and C₁-C₆ alkoxy; and

R⁴ is independently, at each occurrence, selected from the group consisting of H, halo, CN, and C₁-C₆ alkyl.

In yet another embodiment, X is CH; and Y is NH. In still another embodiment, Z is CR⁴; and W is CH or N.

In still another embodiment, R¹ is C₁-C₃ alkyl or C₁-C₃ haloalkyl. In an embodiment, R¹ is C₁-C₃ alkyl. In another embodiment, R¹ is C₁-C₃ haloalkyl.

In yet another embodiment, R² is H or C₁-C₃ alkyl. In still another embodiment, R² is H. In an embodiment, R² is C₁-C₃ alkyl.

In another embodiment, R³ is selected from the group consisting of H, C₁-C₃ alkyl, and C₁-C₃ alkoxy. In yet another embodiment, R³ is H. In still another embodiment, R³ is C₁-C₃ alkyl. In another embodiment, R³ is C₁-C₃ alkoxy.

In an embodiment, R⁴ is independently, at each occurrence, halo or CN. In another embodiment, R⁴ is halo. In yet another embodiment, R⁴ is CN.

In still another embodiment, the compound of Formula I is a compound of Formula Ib:

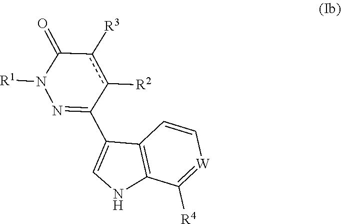

or a pharmaceutically acceptable salt thereof;

wherein W is N or CH.

In an embodiment, the compound of Formula I is a compound of Formula Ic:

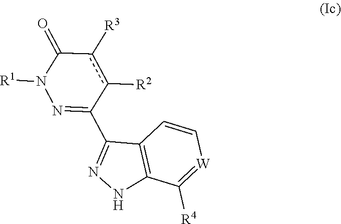

or a pharmaceutically acceptable salt thereof;

wherein W is N or CH.

In an embodiment of Formulae Ia and Ib, R⁴ is halo or CN.

In another embodiment of Formulae Ia and Ib

W is N or CH;

R² is H or C₁-C₃ alkyl;

R³ is selected from the group consisting of H, C₁-C₃ alkyl, and C₁-C₃ alkoxy; and

R⁴ is halo or CN.

In another embodiment, the compound of Formula I is selected from the group consisting of a compound in Table 1.

TABLE 1

| Compound | |
|---|---|
| No. | Structure |
| 001 | 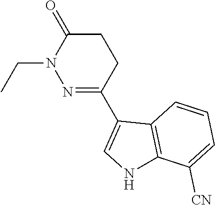 [SMILES: CCN1N=C(c2c[nH]c3c(C#N)cccc23)CCC1=O] |
| 002 | 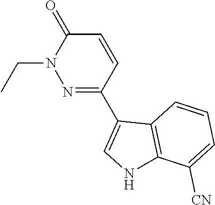 [SMILES: CCn1nc(-c2c[nH]c3c(C#N)cccc23)ccc1=O] |
| 003 | 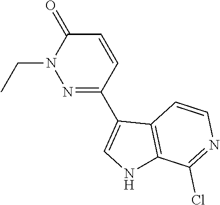 [SMILES: CCn1nc(-c2c[nH]c3c(Cl)nccc23)ccc1=O] |
| 004 | 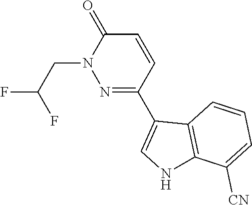 [SMILES: N#Cc1cccc2c(-c3ccc(=O)n(CC(F)F)n3)c[nH]c12] |
| 005 | 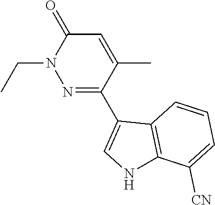 [SMILES: CCn1nc(-c2c[nH]c3c(C#N)cccc23)c(C)cc1=O] |
| 006 | 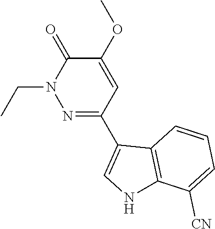 [SMILES: CCn1nc(-c2c[nH]c3c(C#N)cccc23)cc(OC)c1=O] |

or a pharmaceutically acceptable salt thereof.

In another aspect, provided herein is a compound of Formula II:

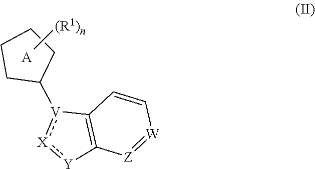

or a pharmaceutically acceptable salt thereof;

wherein

[symbol] is an optional double bond;

A is a 5-membered heteroaryl;

V is C, CH, or N;

X is CH or N;

Y is CH, N, NH, O, or S;

Z and W are each independently CR² or N;

R¹ is independently, at each occurrence, selected from the group consisting of C₁-C₆ alkyl, C₁-C₆ haloalkyl, C₁-C₆ alkoxy, C₃-C₁₀ cycloalkyl, 3-10 membered heterocycloalkyl, C₆-C₁₀ aryl, and 5-10 membered heteroaryl, wherein alkyl, cycloalkyl, heterocycloalkyl, aryl, and heteroaryl are each optionally substituted by one, two, or three R³;

R² is independently, at each occurrence, selected from the group consisting of H, halo, CN, OH, NH₂, NH(C₁-C₆ alkyl), N(C₁-C₆ alkyl)₂, C₁-C₆ alkyl, C₁-C₆ haloalkyl, C₁-C₆ alkyl-OH, and C₁-C₆ alkoxy, provided that when both Z and W are CR², at least one R² is CN; R³ is independently, at each occurrence, selected from the group consisting of halo, CN, OH, SH, NH₂, C₁-C₆ haloalkyl, and C₁-C₆ alkoxy; and

n is 1, 2, or 3.

In an embodiment of Formula II,

[symbol] is an optional double bond;

A is a 5-membered heteroaryl;

V is CH or C;

X is CH or N;

Y is CH, N, NH, O, or S;

Z and W are each independently CH, CR², or N;

R¹ is independently, at each occurrence, selected from the group consisting of C₁-C₆ alkyl, C₁-C₆ haloalkyl, C₁-C₆ alkoxy, C₃-C₁₀ cycloalkyl, and 3-10 membered heterocycloalkyl, wherein alkyl, cycloalkyl, and heterocycloalkyl are each optionally substituted by one, two, or three R³;

R² is independently, at each occurrence, selected from the group consisting of H, halo, CN, C₁-C₆ alkyl, C₁-C₆ haloalkyl, and C₁-C₆ alkoxy; R³ is independently, at each occurrence, selected from the group consisting of halo, CN, OH, SH, NH₂, C₁-C₆ haloalkyl, and C₁-C₆ alkoxy; and

n is 1, 2, or 3.

In an embodiment of Formula II,

A is pyrazole;

V is C;

X is CH;

Y is NH;

Z and W are each independently CH, CR², or N;

R¹ is independently, at each occurrence, selected from the group consisting of C₁-C₆ alkyl, C₃-C₁₀ cycloalkyl, and 3-10 membered heterocycloalkyl, wherein alkyl and cycloalkyl are each optionally substituted by one R³;

R² is independently, at each occurrence, selected from the group consisting of halo, CN, C₁-C₆ alkyl, and C₁-C₆ haloalkyl;

R³ is independently, at each occurrence, selected from the group consisting of halo, OH, SH, and NH₂; and

n is 1 or 2.

In another embodiment, the compound of Formula II is a compound of Formula IIa:

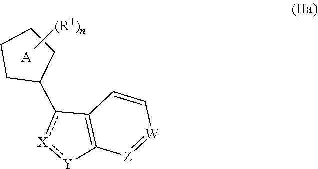

or a pharmaceutically acceptable salt thereof.

In another embodiment, A is pyrazole.

In yet another embodiment, X is CH; and Y is NH. In still another embodiment, Z is CR²; and W is CH or N.

In an embodiment, R¹ is independently, at each occurrence, C₁-C₆ alkyl or C₃-C₁₀ cycloalkyl, wherein alkyl and cycloalkyl are each optionally substituted by one R³. In another embodiment, R¹ is C₁-C₆ alkyl optionally substituted by one R³. In yet another embodiment, R¹ is C₃-C₁₀ cycloalkyl optionally substituted by one R³.

In still another embodiment, R² is independently, at each occurrence, halo or CN. In an embodiment, R² is halo. In another embodiment, R² is CN.

In yet another embodiment, R³ is independently, at each occurrence, halo or OH. In still another embodiment, R³ is halo. In an embodiment, R³ is OH.

In another embodiment, n is 1 or 2. In yet another embodiment, n is 1. In still another embodiment n is 2.

In an embodiment, the compound of Formula II is a compound of Formula IIb:

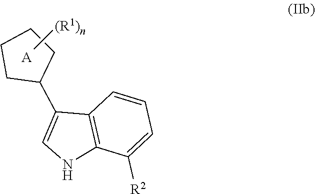

or a pharmaceutically acceptable salt thereof.

In another embodiment, the compound of Formula II is a compound of Formula IIc:

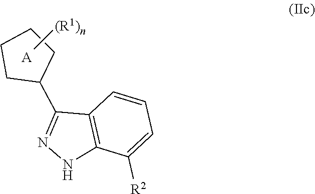

or a pharmaceutically acceptable salt thereof.

In yet another embodiment, the compound of Formula ii is a compound of Formula IId:

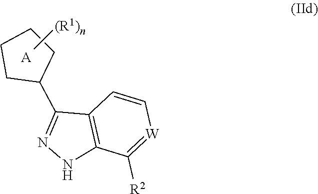

or a pharmaceutically acceptable salt thereof;

wherein W is N or CH.

In still another embodiment, the compound of Formula II is a compound of Formula IIe:

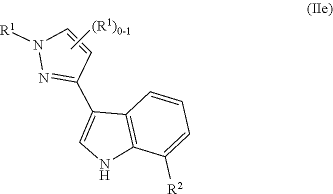

or a pharmaceutically acceptable salt thereof.

In an embodiment of Formulae IIa, IIb, IIc, IId, or IIe,

A is pyrazole;

R¹ is independently, at each occurrence, selected from the group consisting of C₁-C₆ alkyl and C₃-C₁₀ cycloalkyl, wherein alkyl and cycloalkyl are each optionally substituted by OH;

R² is halo or CN; and

n is 1 or 2.

In an embodiment, the compound of Formula II is selected from the group consisting of a compound in Table 2.

TABLE 2

| Compound No. | Structure |
|---|---|
| 007 | 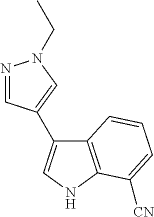 [SMILES: CCn1cc(-c2c[nH]c3c(C#N)cccc23)cn1] |
| 008 | 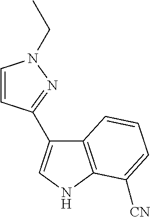 [SMILES: CCn1ccc(-c2c[nH]c3c(C#N)cccc23)n1] |
| 009 | 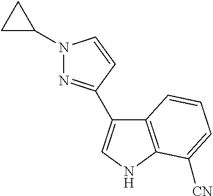 [SMILES: N#Cc1cccc2c(-c3ccn(C4CC4)n3)c[nH]c12] |
| 010 | 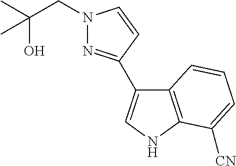 [SMILES: CC(C)(O)Cn1ccc(-c2c[nH]c3c(C#N)cccc23)n1] |
| 011 | 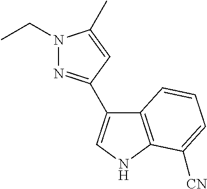 [SMILES: CCn1nc(-c2c[nH]c3c(C#N)cccc23)cc1C] |
| 012 | 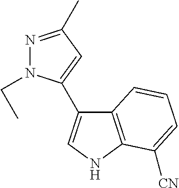 [SMILES: CCn1nc(C)cc1-c1c[nH]c2c(C#N)cccc12] |

or a pharmaceutically acceptable salt thereof.

In an aspect, provided herein is a pharmaceutical composition comprising a compound provided herein, or a pharmaceutically acceptable salt thereof, and a pharmaceutically acceptable carrier.

In one embodiment, the disclosed compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ⁶Cl, ¹⁸F, ¹²³I, ¹²⁵I, ¹³N, ¹⁵N, ¹⁵O, ¹⁷O, ¹⁸O, ³²P, and ³⁵S. In another embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, the compounds described herein include a 2H (i.e., deuterium) isotope.

In still another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

The specific compounds described herein, and other compounds encompassed by one or more of the Formulas described herein having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as described herein are modified by the use of appropriate reagents and conditions, for the introduction of the various moieties found in the Formulas as provided herein. Compounds described herein are synthesized using any suitable procedures starting from compounds that are available from commercial sources, or are prepared using procedures described herein.

### Methods of Treatment

The compounds disclosed herein can be used in a method of treating a disease or condition in a subject, said method comprising administering to the subject a compound provided herein, or a pharmaceutical composition comprising the compound, and a pharmaceutically acceptable carrier.

In still another aspect, provided herein is a method of treating cancer in a subject in need thereof, comprising administering to the subject a therapeutically effective amount of a compound of Formula I or Formula II.

In an embodiment, the cancer is selected from hematological cancers, sarcomas, lung cancers, gastrointestinal cancers, genitourinary tract cancers, liver cancers, bone cancers, nervous system cancers, gynecological cancers, and skin cancers. In an embodiment, the cancer is prostate cancer. In another embodiment, the cancer is ovarian cancer.

In another embodiment, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet another embodiment, the lung cancer is non-small cell lung cancer (NSCLC). In still another embodiment, the lung cancer is adenocarcinoma.

In an embodiment, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In an embodiment, the gastrointestinal cancer is colorectal cancer.

In another embodiment, the cancer is a carcinoma. In yet another embodiment, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still another embodiment, the cancer is a hematopoietic malignancy. In an embodiment, the hematopoietic malignancy is selected from multiple myeloma, acute myelogenous leukemia, and myeloproliferative neoplasms.

In another embodiment, the cancer is a neoplasm. In yet another embodiment, the neoplasm is glioblastoma or sarcomas.

In an embodiment, the cancer is selected from the group consisting of hematological cancers, sarcomas, lung cancers, gastrointestinal cancers, genitourinary tract cancers, liver cancers, bone cancers, nervous system cancers, gynecological cancers, and skin cancers.

In an embodiment, the cancer is selected from the group consisting of pancreatic cancer, cervical cancer, colon cancer, ovarian cancer, breast cancer, pancreatic cancer, carcinoma, and adenocarcinoma.

In another embodiment, the cancer is pancreatic cancer. In yet another embodiment, the cancer is a solid tumor.

In an aspect, provided herein is a method of treating a neurodegenerative disorder in a subject in need thereof comprising administering to the subject a therapeutically effective amount of a compound of Formula I or II, or a pharmaceutically acceptable salt thereof.

In an embodiment, the neurodegenerative disorder is an x-linked recessive disorder. In another embodiment, the neurodegenerative disorder is spinal bulbar muscular atrophy (SBMA).

In another aspect, provided herein is a method of modulating androgen receptor (AR) activity in a subject in need thereof comprising administering to the subject a therapeutically effective amount of a compound of Formula I or II, or a pharmaceutically acceptable salt thereof.

In an embodiment, the androgen receptor (AR) undergoes allosteric modulation. In another embodiment, modulating androgen receptor (AR) activity treats spinal bulbar muscular atrophy (SBMA) in the subject.

In an embodiment of the methods, the subject is human.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

### Administration/Dosage/Formulations

In another aspect, provided herein is a pharmaceutical composition comprising at least one compound provided herein, together with a pharmaceutically acceptable carrier. Actual dosage levels of the active ingredients in the pharmaceutical compositions discussed herein may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated as pharmaceutical compositions using one or more pharmaceutically acceptable excipients or carriers. In one embodiment, the pharmaceutical compositions comprise a therapeutically effective amount of a disclosed compound and a pharmaceutically acceptable carrier.

Routes of administration of any of the compositions disclosed herein include oral, nasal, rectal, intravaginal, parenteral, buccal, sublingual or topical. The compounds disclosed herein may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present application as set forth.

### EXAMPLES

### Example A: Synthetic Procedures

### Abbrevations

ACN acetonitrile
AcOH acetic acid
DCM dichloromethane
DMF dimethylformamide
dppf 1,1′-bis(diphenylphosphino)ferrocene
EtOAc ethyl acetate
MeOH methanol
MTBE methyl tert-butyl ether
PE petroleum ether
TEA triethylamine

### Intermediate A148

### 3-bromo-1-ethylpyrazole

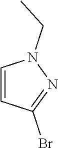

Step 1: To a mixture of 3-nitro-1H-pyrazole (4 g, 35.374 mmol) in DMF (10 mL) was added NaH (1.7 g, 70.833 mmol) at 0° C. under nitrogen atmosphere. The resulting mixture was stirred at 0° C. for 30 min. To the above mixture was added ethyl iodide (11 g, 70.721 mmol) at 0° C. The resulting mixture was stirred at room temperature for 2 h. The resulting mixture was extracted with EtOAc. The combined organic layers were washed with brine, dried over anhydrous Na₂SO₄. After filtration, the filtrate was concentrated under reduced pressure. The residue was purified by flash column chromatography with 0-50% ethyl acetate in PE to afford 1-ethyl-3-nitropyrazole (3.3 g, 66.1%) as a yellow oil. MS m/z 142.1 [M+1]⁺

Step 2: To a mixture of 1-ethyl-3-nitropyrazole (3.1 g, 21.966 mmol) in MeOH (15 mL) was added Pd/C (1.5 g). The resulting mixture was stirred at room temperature for 4 h under hydrogen atmosphere. The resulting mixture was filtered, the filter cake was washed with MeOH. The filtrate was concentrated under reduced pressure. This resulted in 1-ethylpyrazol-3-amine (2.8 g, crude) as a yellow oil. MS m/z 112.2 [M+1]⁺

Step 3: To a mixture of 1-ethylpyrazol-3-amine (1.5 g, 13.496 mmol) in acetonitrile (10 mL) was added CuBr (1.94 g, 13.524 mmol) under nitrogen atmosphere. To the above mixture was added t-BuONO (1.39 g, 13.496 mmol) at 0° C. The resulting mixture was stirred at 50° C. overnight. The resulting mixture was extracted with EtOAc. The combined organic layers were washed with brine, dried over anhydrous Na₂SO₄. After filtration, the filtrate was concentrated under reduced pressure. The residue was purified by flash column chromatography with 0-50% ethyl acetate in PE to afford 3-bromo-1-ethylpyrazole (300 mg, 12.7%) as a yellow oil. ¹H NMR (400 MHz, DMSO-de) δ 7.76 (d, J=2.4 Hz, 1H), 6.35 (d, J=2.4 Hz, 1H), 4.18-4.06 (m, 2H), 1.38-1.33 (m, 3H).

### Intermediate A150

### 3-bromo-1-cyclopropylpyrazole

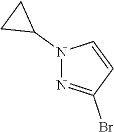

To a mixture of 3-bromo-1H-pyrazole (1 g, 6.804 mmol) and cyclopropylboronic acid (1.17 g, 13.608 mmol) in ACN (15 mL) were added TEA (1.38 g, 13.608 mmol), Cu(OAc)₂ (1.24 g, 6.804 mmol) and 4 A MS (1 g). The resulting mixture was stirred at 80° C. under nitrogen atmosphere for 2 h. The resulting mixture was concentrated under vacuum. The resulting mixture was extracted with EtOAc. The combined organic layers were washed with brine, dried over anhydrous Na₂SO₄. After filtration, the filtrate was concentrated under reduced pressure. The residue was purified by flash column chromatography with 0-50% ethyl acetate in PE to afford 3-bromo-1-cyclopropylpyrazole (97 mg, 7.61%) as a colorless oil. MS m/z 187.0 [M+1]⁺

### Intermediate A151

### 1-(3-bromopyrazol-1-yl)-2-methylpropan-2-ol

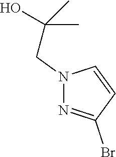

To a mixture of 3-bromo-1H-pyrazole (800 mg, 5.443 mmol) in DMF (20 mL) was added K₂CO₃ (2.77 g, 20.043 mmol) and 2-propanol, 1-chloro-2-methyl- (1.89 g, 17.418 mmol). The resulting mixture was stirred at 35° C. overnight. The resulting mixture was extracted with EtOAc. The combined organic layers were washed with brine, dried over anhydrous Na₂SO₄. After filtration, the filtrate was concentrated under reduced pressure. The residue was purified by reverse flash chromatography with 0-50% ACN in H₂O to afford 1-(3-bromopyrazol-1-yl)-2-methylpropan-2-ol (280 mg, 33.81%) as a colorless oil. MS m/z 219.0 [M+1]⁺

### Intermediate A152 and Intermediate A153

### 3-bromo-1-ethyl-5-methylpyrazole and 5-bromo-1-ethyl-3-methyl-1H-pyrazole

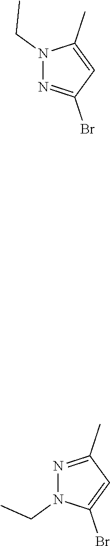

To a mixture of 3-bromo-5-methyl-1H-pyrazole (1 g, 6.211 mmol) in DMF (25 mL) was added K₂CO₃ (3 g, 21.707 mmol) and ethyl iodide (2.91 g, 18.658 mmol). The resulting mixture was stirred at 35° C. for 1 h. The resulting mixture was extracted with EtOAc. The combined organic layers were washed with brine, dried over anhydrous Na₂SO₄. After filtration, the filtrate was concentrated under reduced pressure. The residue was purified by flash column chromatography with 0-50% ethyl acetate in PE to afford a mixture of 3-bromo-1-ethyl-5-methylpyrazole and 5-bromo-1-ethyl-3-methyl-1H-pyrazole (800 mg, 68.13%) as a light-yellow oil. MS m/z 189.0 [M+1]⁺

### Intermediate A155

### 6-chloro-2-ethyl-4,5-dihydropyridazin-3-one

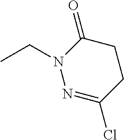

To a mixture of 6-chloro-2-ethylpyridazin-3-one (600 mg, 3.783 mmol) in AcOH (5 mL) was added Zn (1.48 g, 22.7 mmol). The resulting mixture was stirred at room temperature for 4 h. The resulting mixture was concentrated under vacuum. The residue was purified by flash column chromatography with 0-50% ethyl acetate in PE to afford 6-chloro-2-ethyl-4,5-dihydropyridazin-3-one (350 mg, 57.6%) as a yellow oil. MS m/z 161.0 [M+1]⁺

### Intermediate A156

### 6-chloro-2-ethylpyridazin-3-one

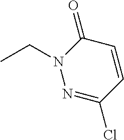

To a mixture of 6-chloro-2H-pyridazin-3-one (3 g, 22.983 mmol) in DMF (5 mL) were added K₂CO₃ (4764 mg, 34.475 mmol). The resulting mixture was stirred at room temperature for 30 min. To the above mixture was added ethyl iodide (5.10 g, 32.692 mmol) at 0° C. The resulting mixture was stirred at room temperature overnight. The resulting mixture was extracted with EtOAc. The combined organic layers were washed with brine, dried over anhydrous Na₂SO₄. After filtration, the filtrate was concentrated under reduced pressure. The residue was purified by flash column chromatography with 0-50% ethyl acetate in PE to afford 6-chloro-2-ethylpyridazin-3-one (2 g, 54.87%) as a yellow oil. MS m/z 159.0 [M+1]⁺

### Intermediate A157

### 6-chloro-2-(2,2-difuoroethyl)pyridazin-3-one

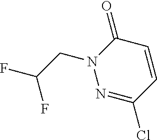 [SMILES: O=c1ccc(Cl)nn1CC(F)F]

To a mixture of 6-chloro-2H-pyridazin-3-one (2 g, 15.322 mmol) in DMF (5 mL) were added K₂CO₃ (6.35 g, 45.966 mmol) and 1,1-difluoro-2-iodoethane (10.29 g, 53.627 mmol) at 0° C. The resulting mixture was stirred at room temperature for 16 h. The resulting mixture was extracted with EtOAc. The combined organic layers were washed with brine, dried over anhydrous Na₂SO₄. After filtration, the filtrate was concentrated under reduced pressure. The residue was purified by flash column chromatography with 0-50% ethyl acetate in PE to afford 6-chloro-2-(2,2-difluoroethyl)pyridazin-3-one (325 mg, 10.90%) as a white solid. MS m/z 195.0 [M+1]⁺

### Intermediate A158

### 6-chloro-2-ethyl-5-methylpyridazin-3-one

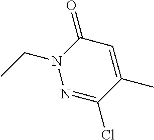

To a mixture of 6-chloro-5-methyl-2H-pyridazin-3-one (500 mg, 3.459 mmol) in DMF (15 mL) was added K₂CO₃ (717 mg, 5.188 mmol) and ethyl iodide (647 mg, 4.151 mmol) at 0° C. The resulting mixture was stirred at room temperature for 1 h. The resulting mixture was extracted with EtOAc. The combined organic layers were washed with brine, dried over anhydrous Na₂SO₄. After filtration, the filtrate was concentrated under reduced pressure. The residue was purified by flash column chromatography with 0-60% ethyl acetate in PE to afford 6-chloro-2-ethyl-5-methylpyridazin-3-one (490 mg, 73.87%) as a yellow oil. MS m/z 173.0 [M+1]4

### Intermediate A159

### 6-chloro-2-ethyl-4-methoxypyridazin-3-one

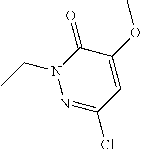

Step 1: To a mixture 4-bromo-6-chloro-2H-pyridazin-3-one (1 g, 4.775 mmol) in DMF (5 mL) were added K₂CO₃ (988 mg, 7.163 mmol) and ethyl iodide (936 mg, 6.00 mmol). The resulting mixture was stirred at room temperature for 16 h. The resulting mixture was extracted with EtOAc. The combined organic layers were washed with brine, dried over anhydrous Na₂SO₄. After filtration, the filtrate was concentrated under reduced pressure. The residue was purified by flash column chromatography with 0-50% ethyl acetate in PE to afford 4-bromo-6-chloro-2-ethylpyridazin-3-one (190 mg, 16.76%) as a yellow solid. MS m/z 236.9 [M+1]⁺

Step 2: To a mixture of 4-bromo-6-chloro-2-ethylpyridazin-3-one (200 mg, 0.842 mmol) in CH₃OH (3 mL) were added sodium methoxide (182 mg, 3.369 mmol). The resulting mixture was stirred at room temperature for 2 h. The mixture was concentrated under vacuum. The residue was purified by flash column chromatography with 0-5% MeOH in DCM to afford 6-chloro-2-ethyl-4-methoxypyridazin-3-one (130 mg, 81.81%) as a yellow oil. MS m/z 189.0 [M+1]⁺

### Intermediate B51

### 7-chloro-1H-pyrrolo[2,3-c]pyridin-3-ylboronic acid

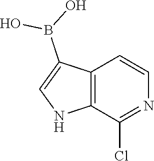 [SMILES: OB(O)c1c[nH]c2c(Cl)nccc12]

To a mixture of 7-chloro-1H-pyrrolo[2,3-c]pyridine (200 mg, 1.311 mmol) in MTBE (5 mL) were added 4,4,5,5-tetramethyl-2-(tetramethyl-1,3,2-dioxaborolan-2-yl)-1,3,2-dioxaborolane (666 mg, 2.622 mmol), Bis(1,5-cyclooctadiene)dimethoxydiiridium (17 mg, 0.026 mmol) and dtbpy (18 mg, 0.066 mmol). The resulting mixture was stirred at 80° C. for 1 h under nitrogen atmosphere. The reaction was quenched with MeOH at 0° C. The resulting mixture was concentrated under vacuum. This resulted in 7-chloro-1H-pyrrolo[2,3-c]pyridin-3-ylboronic acid (200 mg, crude) as a brown solid. MS m/z 197.0 [M+1]⁺

### Compound 001

3-(1-ethyl-6-oxo-4,5-dihydropyridazin-3-yl)-1H-indole-7-carbonitrile To a mixture of 6-chloro-2-ethyl-4,5-dihydropyridazin-3-one (130 mg, 0.809 mmol) and tert-butyl 7-cyano-3-(4,4,5,5-tetramethyl-1,3,2-dioxaborolan-2-yl)indole-1-carboxylate (298 mg, 0.809 mmol) in dioxane (2 mL) and H₂O (0.2 mL) were added Pd(dppf)Cl₂ (59 mg, 0.081 mmol) and K₂CO₃ (224 mg, 1.618 mmol). The resulting mixture was stirred at 100° C. overnight under nitrogen atmosphere. The resulting mixture was concentrated under vacuum. The residue was purified by Prep-HPLC [Column: XBridge Prep OBD C18 Column, 30×150 mm 5 um; Mobile Phase A: water (10 mmol/L NH₄HCO₃), Mobile Phase B: ACN; Flow rate: 60 mL/min; Gradient: 20 B to 60 B in 8 min; 254/220 nm] to afford 3-(1-ethyl-6-oxo-4,5-dihydropyridazin-3-yl)-1H-indole-7-carbonitrile (35.8 mg, 16.61%) as a white solid.

Compound 002

### 3-(1-ethyl-6-oxopyrdazin-3-yl)-1H-indole-7-carbonitrile

To a mixture of 6-chloro-2-ethylpyridazin-3-one (500 mg, 3.153 mmol) and tert-butyl 7-cyano-3-(4,4,5,5-tetramethyl-1,3,2-dioxaborolan-2-yl)indole-1-carboxylate (1.16 g, 3.153 mmol) in dioxane (5 mL) and H₂O (0.5 mL) were added K₂CO₃ (871 mg, 6.306 mmol) and Pd(dppf)Cl₂ (230 mg, 0.315 mmol). The resulting mixture was stirred at 100° C. for 1 h under nitrogen atmosphere. The mixture was concentrated under vacuum. The residue was purified by reverse phase flash chromatography with 0-40% ACN in H₂O to afford 3-(1-ethyl-6-oxopyridazin-3-yl)-1H-indole-7-carbonitrile (42.7 mg, 5.12%) as a white solid.

### Compound 003

6-[7-chloro-1H-pyrrolo[2,3-c]pyridin-3-yl]-2-ethylpyridazin-3-one To a mixture of 6-chloro-2-ethylpyridazin-3-one (200. mg, 1.261 mmol) and 7-chloro-1H-pyrrolo[2,3-c]pyridin-3-ylboronic acid (495 mg, 2.522 mmol) in dioxane (4 mL) and H₂O (0.4 mL) were added Pd(dppf)Cl₂ (185 mg, 0.252 mmol) and K₂CO₃ (523 mg, 3.783 mmol). The resulting mixture was stirred at 100° C. overnight under nitrogen atmosphere. The resulting mixture was concentrated under vacuum. The residue was purified by Prep-HPLC [Column: XBridge Prep OBD C18 Column, 30×150 mm 5 um; Mobile Phase A: water (10 mmol/L NH₄HCO₃), Mobile Phase B: ACN; Flow rate: 60 mL/min; Gradient: 20 B to 45 B in 8 min, 254/220 nm] to afford 6-[7-chloro-1H-pyrrolo[2,3-c]pyridin-3-yl]-2-ethylpyridazin-3-one (12.8 mg, 3.69%) as a white solid.

### Compound 004

3-[1-(2,2-difluoroethyl)-6-oxopyridazin-3-yl]-1H-indole-7-carbonitrile To a mixture of 6-chloro-2-(2,2-difluoroethyl)pyridazin-3-one (250 mg, 1.285 mmol) and tert-butyl 7-cyano-3-(4,4,5,5-tetramethyl-1,3,2-dioxaborolan-2-yl)indole-1-carboxylate (473 mg, 1.285 mmol) in dioxane (3 mL) and H₂O (0.3 mL) were added K₂CO₃(355 mg, 2.57 mmol) and Pd(dppf)Cl₂ (94 mg, 0.128 mmol). The resulting mixture was stirred at 100° C. for 16 h under nitrogen atmosphere. The mixture was concentrated under vacuum. The residue was purified by Prep-HPLC [Column: Xselect CSH OBD Column 30*150 mm 5 um; Mobile Phase A: water (10 mmol/L NH₄HCO₃), Mobile Phase B: ACN; Flow rate: 60 mL/min; Gradient: 20 B to 70 B in 8 min, 254 nm] to afford 3-[1-(2,2-difluoroethyl)-6-oxopyridazin-3-yl]-1H-indole-7-carbonitrile (82.1 mg, 21.28%) as a white solid.

### Compound 005

### 3-(1-ethyl-4-methyl-6-oxopyridazin-3-yl)-1H-indole-7-carbonitrile

To a mixture of 6-chloro-2-ethyl-5-methylpyridazin-3-one (400 mg, 2.317 mmol) and tert-butyl 7-cyano-3-(4,4,5,5-tetramethyl-1,3,2-dioxaborolan-2-yl)indole-1-carboxylate (1.28 g, 3.476 mmol) in dioxane (10 mL) and H₂O (1 mL) were added K₂CO₃ (960 mg, 6.952 mmol) and Pd(dppf)Cl₂ (169 mg, 0.232 mmol). The resulting mixture was stirred at 100° C. overnight under nitrogen atmosphere. The resulting mixture was concentrated under reduced pressure. The residue was purified by reverse phase flash chromatography with 0-40% ACN in H₂O to afford 3-(1-ethyl-4-methyl-6-oxopyridazin-3-yl)-1H-indole-7-carbonitrile (20.4 mg, 3.14%) as a white solid.

### Compound 006

3-(1-ethyl-5-methoxy-6-oxopyridazin-3-yl)-1H-indole-7-carbonitrile To a mixture of 6-chloro-2-ethyl-4-methoxypyridazin-3-one (130 mg, 0.689 mmol) and tert-butyl 7-cyano-3-(4,4,5,5-tetramethyl-1,3,2-dioxaborolan-2-yl)indole-1-carboxylate (304 mg, 0.827 mmol) in dioxane (3 mL) and H₂O (0.3 mL) were added K₂CO₃ (190 mg, 1.379 mmol) and Pd(dppf)Cl₂ (50 mg, 0.069 mmol). The resulting mixture was stirred at 100° C. for 16 h under nitrogen atmosphere. The mixture was concentrated under vacuum. The residue was purified by Prep-HPLC [Column: YMC-Actus Triart C18 ExRS, 30 mm×150 mm, 5 um; Mobile Phase A: water (10 mmol/L NH₄HCO₃), Mobile Phase B: ACN; Flow rate: 60 mL/min; Gradient: 35 B to 40 B in 11 min, 254 nm] to afford 3-(1-ethyl-5-methoxy-6-oxopyridazin-3-yl)-1H-indole-7-carbonitrile (15 mg, 7.39%) as a white solid.

### Compound 007

3-(1-ethylpyrazol-4-yl)-1H-indole-7-carbonitrile To a mixture of 4-bromo-1-ethylpyrazole (150 mg, 0.857 mmol) and tert-butyl 7-cyano-3-(4,4,5,5-tetramethyl-1,3,2-dioxaborolan-2-yl)indole-1-carboxylate (378.7 mg, 1.028 mmol) in dioxane (15 mL) and H₂O (1.5 mL) were added K₂CO₃ (236.88 mg, 1.714 mmol) and Pd(dppf)Cl₂ (62.71 mg, 0.086 mmol). The resulting mixture was stirred at 100° C. under nitrogen atmosphere overnight. The resulting mixture was concentrated under reduced pressure. The residue was purified by reverse flash chromatography with 0-60% ACN in H₂O to afford 3-(1-ethylpyrazol-4-yl)-1H-indole-7-carbonitrile (16.3 mg, 8.04%) as a white solid.

### Compound 008

3-(1-ethylpyrazol-3-yl)-1H-indole-7-carbonitrile To a mixture of 3-bromo-1-ethylpyrazole (200 mg, 1.143 mmol) and tert-butyl 7-cyano-3-(4,4,5,5-tetramethyl-1,3,2-dioxaborolan-2-yl)indole-1-carboxylate (421 mg, 1.143 mmol) in dioxane (4 mL) and H₂O (0.4 mL) were added Pd(dppf)Cl₂ (83.61 mg, 0.114 mmol) and K₂CO₃ (316 mg, 2.285 mmol). The resulting mixture was stirred at 100° C. overnight under nitrogen atmosphere. The resulting mixture was concentrated under vacuum. The residue was purified by reverse phase flash chromatography with 0-60% ACN in H₂O to afford 3-(1-ethylpyrazol-3-yl)-1H-indole-7-carbonitrile (26.6 mg, 9.85%) as an off-white solid.

### Compound 009

3-(1-cyclopropylpyrazol-3-yl)-1H-indole-7-carbonitrile To a mixture of 3-bromo-1-cyclopropylpyrazole (96 mg, 0.513 mmol) and tert-butyl 7-cyano-3-(4,4,5,5-tetramethyl-1,3,2-dioxaborolan-2-yl)indole-1-carboxylate (283 mg, 0.77 mmol) in dioxane (5 mL) and H₂O (0.5 mL) were added K₂CO₃ (212 mg, 1.54 mmol) and Pd(dppf)Cl₂ (37 mg, 0.051 mmol). The resulting mixture was stirred at 100° C. overnight under nitrogen atmosphere. The resulting mixture was concentrated under vacuum. The residue was purified by Prep-HPLC [Column: XBridge Prep OBD C18 Column, 30×150 mm 5 um; Mobile Phase A: water (10 mmol/L NH₄HCO₃), Mobile Phase B: ACN; Flow rate: 60 mL/min; Gradient: 33 B to 52 B in 8 min; 254/220 nm] to afford 3-(1-cyclopropylpyrazol-3-yl)-1H-indole-7-carbonitrile (26 mg, 56.54%) as a white solid.

### Compound 010

3-[1-(2-hydroxy-2-methylpropyl)pyrazol-3-yl]-1H-indole-7-carbonitrile To a mixture of 1-(3-bromopyrazol-1-yl)-2-methylpropan-2-ol (200 mg, 0.913 mmol) and tert-butyl 7-cyano-3-(4,4,5,5-tetramethyl-1,3,2-dioxaborolan-2-yl)indole-1-carboxylate (504 mg, 1.369 mmol) in dioxane (10 mL) and H₂O (1 mL) were added K₂CO₃(378 mg, 2.739 mmol) and Pd(dppf)Cl₂ (67 mg, 0.091 mmol). The resulting mixture was stirred at 100° C. overnight under nitrogen atmosphere. The resulting mixture was concentrated under vacuum. The residue was purified by reverse flash chromatography with 0-40% ACN in H₂O to afford 3-[1-(2-hydroxy-2-methylpropyl)pyrazol-3-yl]-1H-indole-7-carbonitrile (16.0 mg, 6.15%) as a white solid.

### Compounds 011 and 012

### 3-(1-ethyl-5-methylpyrazol-3-yl)-1H-indole-7-carbonitrile and 3-(2-ethyl-5-methylpyrazol-3-yl)-1H-indole-7-carbonitrile

To a mixture of 3-bromo-1-ethyl-5-methylpyrazole and 5-bromo-1-ethyl-3-methyl-1H-pyrazole (150 mg, 0.793 mmol) in dioxane (3 mL) and H₂O (0.6 mL) were added tert-butyl 3-boranyl-7-cyanoindole-1-carboxylate (404 mg, 1.587 mmol), K₂CO₃ (329 mg, 2.380 mmol) and Pd(dppf)Cl₂ (54 mg, 0.073 mmol). The resulting mixture was stirred at 80° C. for 16 h under nitrogen atmosphere. The mixture was concentrated under vacuum. The residue was purified by Prep-HPLC [Column: XBridge Prep OBD C18 Column, 30×150 mm 5 um; Mobile Phase A: water (10 mmol/L NH₄HCO₃), Mobile Phase B: ACN; Flow rate: 60 mL/min; Gradient: 35 B to 50 B in 8 min; 254/220 nm] to afford 3-(1-ethyl-5-methylpyrazol-3-yl)-1H-indole-7-carbonitrile (12.8 mg, 3.17%) as a white solid and 3-(2-ethyl-5-methylpyrazol-3-yl)-1H-indole-7-carbonitrile (35.6 mg, 17.67%) as a white solid.

### Example B: Biological Assay

LNCaP cells expressing ARR2PB-FireflyLuc and CMV-RenillaLuc were treated with indicated concentrations of test compounds, enzalutamide (negative control), or DHT (positive control)+/−0.5 nM DHT for 48 h at 37° C. Fluorescent signals were read with the ImageXpress Micro Confocal System. Remaining activity (antagonist mode) was calculated as % Remaining Activity=100×[(Read_{Sample}−Cₐᵥₑ)/(HCₐᵥₑ−LCₐᵥₑ)] where HC is cells treated with 0.5 nM DHT only and LC is cells treated with 10 uM enzalutamide+0.5 nM DHT. Activation (agonist mode) was calculated as % Activation=100×[(ReadSample−LCₐᵥₑ)/(HCₐᵥₑ−LCₐᵥₑ)] where HC is cells treated with 1 uM DHT and LC is cells treated with DMSO. Dose response curves and IC₅₀ values were calculated using non-linear regression analysis in XLfit.

IC₅₀ values for the compounds provided herein are shown in Table 3 below. The designation “A” indicates an IC₅₀ value of >10 μM, “B” indicates an IC₅₀ value between 1 μM and 10 μM; “C” indicates an IC₅₀ value between 100 nM and 1 μM; and “D” indicates an IC₅₀ value of less than 100 nM.

TABLE 3

| No. | Structure | IC₅₀ | ¹H NMR | MS (ESI) |
|---|---|---|---|---|
| 001 | 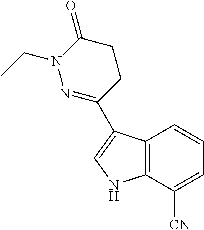 [SMILES: CCN1N=C(c2c[nH]c3c(C#N)cccc23)CCC1=O] | C | ¹H NMR (400 MHz, Methanol- d₄) δ 8.66 (d, J = 8.0 Hz, 1H), 7.86 (s, 1H), 7.61 (d, J = 7.6 Hz, 1H), 7.33-7.24 (m, 1H), 3.99-3.90 (m, 2H), 3.06-2.97 (m, 2H), 2.64-2.56 (m, 2H), 1.39-1.31 (m, 3H). | MS (ESI) calc'd for (C₁₅H₁₄N₄O) [M + 1]⁺, 267.1, found 267.1. |
| 002 | 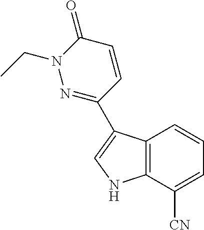 [SMILES: CCn1nc(-c2c[nH]c3c(C#N)cccc23)ccc1=O] | D | ¹H NMR (400 MHz, Methanol- d₄) δ 8.70-8.63 (m, 1H), 8.03 (s, 1H), 7.97 (d, J = 9.6 Hz, 1H), 7.66-7.60 (m, 1H), 7.36-7.27 (m, 1H), 7.02 (d, J = 9.6 Hz, 1H), 4.39-4.29 (m, 2H), 1.54- 1.45 (m, 3H). | MS (ESI) calc'd for (C₁₅H₁₂N₄O) [M + 1]⁺, 265.1, found 265.2. |
| 003 | 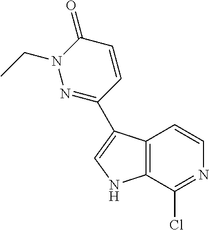 [SMILES: CCn1nc(-c2c[nH]c3c(Cl)nccc23)ccc1=O] | A | ¹H NMR (400 MHz, Methanol- d₄) δ 8.11-8.04 (m, 1H), 7.96- 7.90 (m, 1H), 7.63-7.57 (m, 1H), 7.17 (s, 1H), 7.13-7.06 (m, 1H), 4.42-4.32 (m, 2H), 1.52-1.44 (m, 3H). | MS (ESI) calc'd for (C₁₃H₁₁ClN₄O) [M + 1]⁺, 275.1; found, 275.0. |
| 004 | 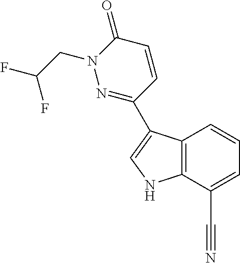 [SMILES: N#Cc1cccc2c(-c3ccc(=O)n(CC(F)F)n3)c[nH]c12] | C | ¹H NMR (300 MHz, DMSO-d₆) δ 12.50 (s, 1H), 8.68-8.59 (m, 1H), 8.33 (s, 1H), 8.17-8.07 (m, 1H), 7.77-7.68 (m, 1H), 7.38-7.26 (m, 1H), 7.13-7.04 (m, 1H), 6.74-6.18 (m, 1H), 4.71-4.54 (m, 2H). | MS (ESI) calc'd for (C₁₅H₁₀F₂N₄O) [M + 1]⁺, 301.0, found 301.0. |
| 005 | 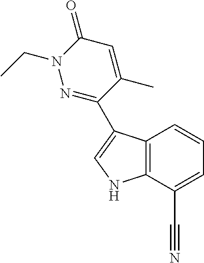 [SMILES: CCn1nc(-c2c[nH]c3c(C#N)cccc23)c(C)cc1=O] | B | ¹H NMR (400 MHz, Methanol- d₄) δ 8.28 (d, J = 8.0 Hz, 1H), 7.82 (s, 1H), 7.64 (d, J = 7.2 Hz, 1H), 7.33-7.25 (m, 1H), 6.96-6.92 (m, 1H), 4.37-4.28 (m, 2H), 2.45-2.40 (m, 3H), 1.50-1.42 (m, 3H). | MS (ESI) calc'd for (C₁₈H₁₄N₄O) [M + 1]⁺, 279.1, found 279.0. |
| 006 | 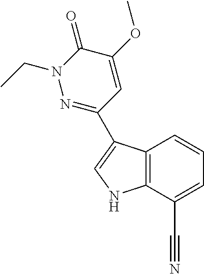 [SMILES: CCn1nc(-c2c[nH]c3c(C#N)cccc23)cc(OC)c1=O] | B | ¹H NMR (400 MHz, DMSO-d₆) δ 12.41 (s, 1H), 8.65 (d, J = 8.0 Hz, 1H), 8.39 (s, 1H), 7.71 (d, J = 7.6 Hz, 1H), 7.38-7.28 (m, 2H), 4.25-4.15 (m, 2H), 3.93 (s, 3H), 1.40-1.32 (m, 3H). | MS (ESI) calc'd for (C₁₈H₁₄N₄O₂) [M + 1]⁺, 295.1; found, 295.0. |
| 007 | 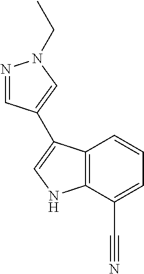 [SMILES: CCn1cc(-c2c[nH]c3c(C#N)cccc23)cn1] | B | ¹H NMR (300 MHz, Methanol- d₄) δ 8.12-8.03 (m, 1H), 7.99 (s, 1H), 7.80 (s. 1H), 7.59- 7.50 (m, 2H), 7.27-7.16 (m, 1H), 4.32-4.18 (m, 2H), 1.56- 1.46 (m, 3H) | MS (ESI) calc'd for (C₁₄H₁₂N₄) [M + 1]⁺, 237.1, found 237.1. |
| 008 | 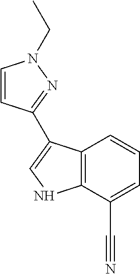 [SMILES: CCn1ccc(-c2c[nH]c3c(C#N)cccc23)n1] | D | ¹H NMR (400 MHz, Methanol- d₄) δ 8.40-8.34 (m, 1H), 7.72 (s, 1H), 7.68 (d, J = 2.4 Hz, 1H), 7.60-7.54 (m, 1H), 7.29-7.20 (m, 1H), 6.58 (d, J = 2.4 Hz, 1H), 4.31-4.21 (m, 2H), 1.57- 1.49 (m, 3H). | MS (ESI) calc'd for (C₁₅H₁₂N₄) [M + 1]⁺, 237.1; found, 237.1. |
| 009 | 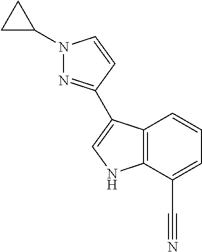 [SMILES: N#Cc1cccc2c(-c3ccn(C4CC4)n3)c[nH]c12] | C | ¹H NMR (400 MHz, Methanol- d₄) δ 8.41-8.34 (m, 1H), 7.76- 7.68 (m, 2H), 7.61-7.54 (m, 1H), 7.30-7.21 (m, 1H), 6.57 (d, J = 2.4 Hz, 1H), 3.76-3.66 (m, 1H), 1.23-1.13 (m, 2H), 1.16-1.04 (m, 2H). | MS (ESI) calc'd for (C₁₅H₁₂N₄) [M + 1]⁺, 249.1, found 249.0. |
| 010 | 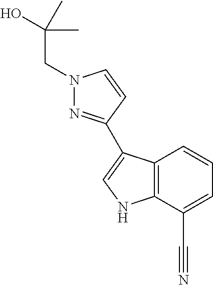 [SMILES: CC(C)(O)Cn1ccc(-c2c[nH]c3c(C#N)cccc23)n1] | A | ¹H NMR (400 MHz, Methanol- d₄) δ 8.43-8.37 (m, 1H), 7.76- 7.67 (m, 2H), 7.61-7.54 (m, 1H), 7.28-7.20 (m, 1H), 6.62 (d, J = 2.4 Hz, 1H), 4.18 (s, 2H), 1.26 (s, 6H). | MS (ESI) calc'd for (C₁₆H₁₆N₄O) [M + 1]⁺, 281.1, found 281.1. |
| 011 | 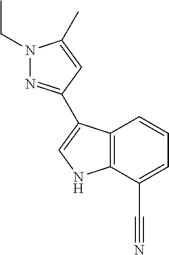 [SMILES: CCn1nc(-c2c[nH]c3c(C#N)cccc23)cc1C] | C | ¹H NMR (400 MHz, Methanol- d₄) δ 8.37-8.30 (m, 1H), 7.68 (s, 1H), 7.56 (d, J = 7.2 Hz, 1H), 7.28-7.20 (m, 1H), 6.38 (s, 1H), 4.24-4.14 (m, 2H), 2.38 (s, 3H), 1.50-1.41 (m, 3H) | MS (ESI) calc'd for (C₁₅H₁₄N₄) [M + 1]⁺, 251.1, found 251.0. |
| 012 | 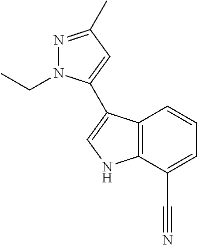 [SMILES: CCn1nc(C)cc1-c1c[nH]c2c(C#N)cccc12] | D | ¹H NMR (400 MHz, Methanol- d₄) δ 7.84 (d, J = 8.0 Hz, 1H), 7.66-7.56 (m, 2H), 7.30-7.22 (m, 1H), 6.23 (s, 1H), 4.17- 4.07 (m, 2H), 2.32 (s, 3H), 1.37-1.29 (m, 3H). | MS (ESI) calc'd for (C₁₅H₁₄N₄) [M + 1]⁺, 251.1, found 251.1. |

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A compound of Formula I: 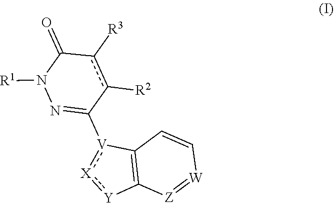
or a pharmaceutically acceptable salt thereof;
wherein [symbol]
is an optional double bond;
V is C, CH, or N;
X is CH or N;
Y is CH, N, NH, O, or S;
Z and W are each independently CR⁴ or N;
R¹ is selected from the group consisting of H, C₁-C₆ alkyl, C₁-C₆ haloalkyl, C₃-C₁₀ cycloalkyl, 3-10 membered heterocycloalkyl, C₆-C₁₀ aryl, and 5-10 membered heteroaryl;
R² is selected from the group consisting of H, C₁-C₆ alkyl, C₁-C₆ haloalkyl, C₁-C₆ alkoxy, C₃-C₁₀ cycloalkyl, 3-10 membered heterocycloalkyl, C₆-C₁₀ aryl, and 5-10 membered heteroaryl;
R³ is selected from the group consisting of H, C₁-C₆ alkyl, C₁-C₆ haloalkyl, C₁-C₆ alkoxy, C₃-C₁₀ cycloalkyl, 3-10 membered heterocycloalkyl, C₆-C₁₀ aryl, and 5-10 membered heteroaryl; and
R⁴ is independently, at each occurrence, selected from the group consisting of H, halo, CN, OH, NH₂, NH(C₁-C₆ alkyl), N(C₁-C₆ alkyl)₂, C₁-C₆ alkyl, C₁-C₆ haloalkyl, C₁-C₆ alkyl-OH, and C₁-C₆ alkoxy.

2. (canceled)

3. The compound of claim 1, wherein [symbol]
is an optional double bond;
V is C or CH;
X is CH;
Y is NH;
Z and W are each independently CR⁴ or N;
R¹ is C₁-C₆ alkyl or C₁-C₆ haloalkyl;
R² is H or C₁-C₆ alkyl;
R³ is selected from the group consisting of H, C₁-C₆ alkyl, and C₁-C₆ alkoxy; and
R⁴ is independently, at each occurrence, selected from the group consisting of H, halo, CN, and C₁-C₆ alkyl.

4. The compound of claim 1, wherein the compound of Formula I is a compound of Formula Ia: 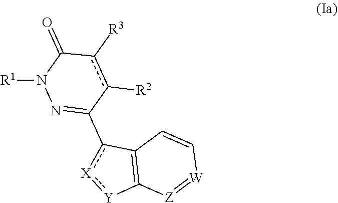
or a pharmaceutically acceptable salt thereof.

5-6. (canceled)

7. The compound of claim 1, wherein R¹ is C₁-C₃ alkyl or C₁-C₃ haloalkyl.

8. The compound of claim 1, wherein R² is H or C₁-C₃ alkyl.

9. The compound of claim 1, wherein R³ is selected from the group consisting of H, C₁-C₃ alkyl, and C₁-C₃ alkoxy.

10. The compound of claim 1, wherein R⁴ is independently, at each occurrence, halo or CN.

11. The compound of claim 1, wherein the compound of Formula I is a compound of Formula Ib: 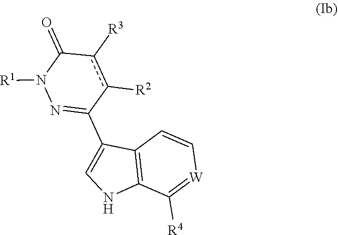
or a pharmaceutically acceptable salt thereof;
wherein W is N or CH.

12. (canceled)

13. The compound of claim 1, wherein the compound of Formula I is selected from the group consisting of 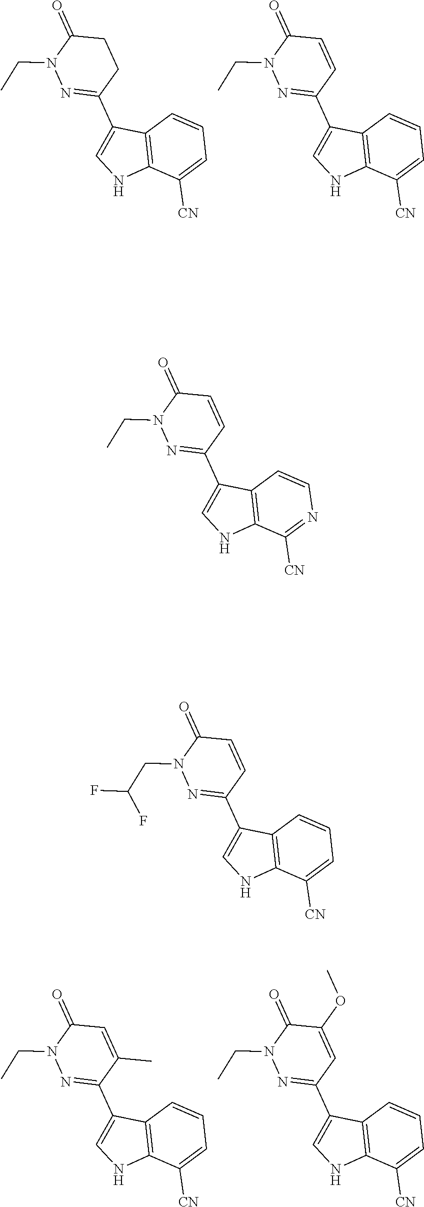
or a pharmaceutically acceptable salt thereof.

14. A compound of Formula II: 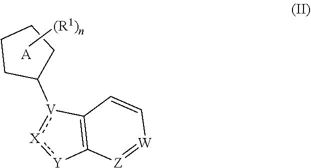
or a pharmaceutically acceptable salt thereof;
wherein [symbol]
is an optional double bond;
A is a 5-membered heteroaryl;
V is C, CH, or N;
X is CH or N;
Y is CH, N, NH, O, or S;
Z and W are each independently CR² or N;
R¹ is independently, at each occurrence, selected from the group consisting of C₁-C₆ alkyl, C₁-C₆ haloalkyl, C₁-C₆ alkoxy, C₃-C₁₀ cycloalkyl, 3-10 membered heterocycloalkyl, C₆-C₁₀ aryl, and 5-10 membered heteroaryl, wherein alkyl, cycloalkyl, heterocycloalkyl, aryl, and heteroaryl are each optionally substituted by one, two, or three R³;
R² is independently, at each occurrence, selected from the group consisting of H, halo, CN, OH, NH₂, NH(C₁-C₆ alkyl), N(C₁-C₆ alkyl)₂, C₁-C₆ alkyl, C₁-C₆ haloalkyl, C₁-C₆ alkyl-OH, and C₁-C₆ alkoxy, provided that when both Z and W are CR², at least one R² is CN;
R³ is independently, at each occurrence, selected from the group consisting of halo, CN, OH, SH, NH₂, C₁-C₆ haloalkyl, and C₁-C₆ alkoxy; and
n is 1, 2, or 3.

15. (canceled)

16. The compound of claim 14, wherein
A is pyrazole;
V is C;
X is CH;
Y is NH;
Z and W are each independently CR² or N;
R¹ is independently, at each occurrence, selected from the group consisting of C₁-C₆ alkyl, C₃-C₁₀ cycloalkyl, and 3-10 membered heterocycloalkyl, wherein alkyl and cycloalkyl are each optionally substituted by one R³;
R² is independently, at each occurrence, selected from the group consisting of halo, CN, C₁-C₆ alkyl, and C₁-C₆ haloalkyl;
R³ is independently, at each occurrence, selected from the group consisting of halo, OH, SH, and NH₂; and
n is 1 or 2.

17-20. (canceled)

21. The compound of claim 14, wherein R¹ is independently, at each occurrence, C₁-C₆ alkyl or C₃-C₁₀ cycloalkyl, wherein alkyl and cycloalkyl are each optionally substituted by one R³.

22. The compound of claim 14, wherein R² is independently, at each occurrence, halo or CN.

23. The compound of claim 14, wherein R³ is independently, at each occurrence, halo or OH.

24. The compound of claim 14, wherein n is 1 or 2.

25. The compound of claim 14, wherein the compound of Formula II is a compound of Formula IIb: 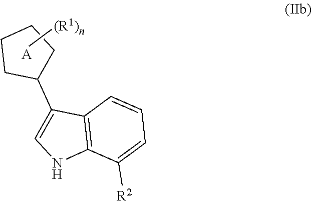
or a pharmaceutically acceptable salt thereof;
wherein A is pyrazole.

26-29. (canceled)

30. The compound of claim 14, wherein the compound of Formula II is selected from the group consisting of 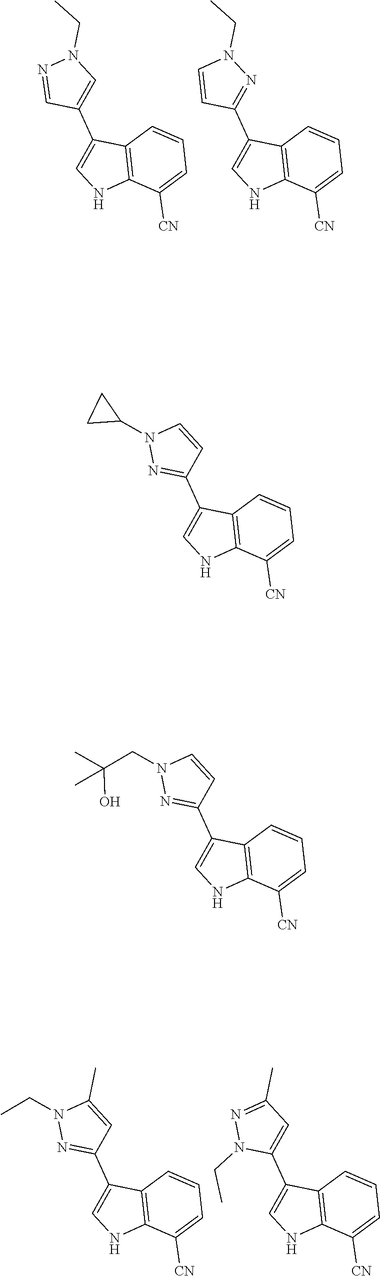
or a pharmaceutically acceptable salt thereof.

31. A pharmaceutical composition comprising a compound of claim 1 and a pharmaceutically acceptable carrier.

32. A method of treating a neurodegenerative disorder in a subject in need thereof comprising administering to the subject a therapeutically effective amount of a compound of claim 1.

33. The method of claim 32, wherein the neurodegenerative disorder is an x-linked recessive disorder.

34. The method of claim 32, wherein the neurodegenerative disorder is spinal bulbar muscular atrophy (SBMA).

35. A method of modulating androgen receptor (AR) activity in a subject in need thereof comprising administering to the subject a therapeutically effective amount of a compound of claim 1.

36. The method of claim 35, wherein the androgen receptor (AR) undergoes allosteric modulation.

37. (canceled)

38. A method of treating cancer in a subject in need thereof comprising administering to the subject a therapeutically effective amount of a compound of claim 1.
